(19) United States
(12) Patent Application Publication
(10) Pub. No.: US 2010/0152719 A1
(43) Pub. Date: Jun. 17, 2010

(54) OPTICAL DEPILATION APPARATUS

(75) Inventor: Shoji Fujikawa, Konan (JP)

Correspondence Address: GREENBLUM & BERNSTEIN, P.L.C., 1950 ROLAND CLARKE PLACE, RESTON, VA, 20191, US

(73) Assignee: PANASONIC ELECTRIC WORKS CO., LTD., Osaka (JP)

(21) Appl. No.: 12/601,054

(22) PCT Filed: May 26, 2008
(86) PCT No.: PCT/JP2008/059653

§ 371 Date: Nov. 20, 2009

(30) Foreign Application Priority Data
May 28, 2007 (JP) 2007-141099

(51) Int. Cl. A61B 18/18

(52) U.S. Cl. 606/9

(57) ABSTRACT

An optical depilation apparatus of high safety is provided, in which the light irradiating the skin during the depilation treatment can be prevented from leaking and entering the eyes, maintenance can be facilitated, and the light for depilation can be prevented from being emitted at an unintended time and entering the eyes. The apparatus for performing depilation by irradiating the skin with light includes: an apparatus body; a light source unit provided at the apparatus body and capable of emitting the light for irradiating the skin; a light shielding pipe having a skin contact surface at an end surface on the side opposite to the apparatus body in the axial direction of the light shielding pipe and is attached detachably to the apparatus body so as to surround a portion of the apparatus body which radiates light emitted from the light source unit; and a control circuit that disables the emission of light from the light source unit in a state in which the light shielding pipe is not attached to the apparatus body.

### TECHNICAL FIELD

The present invention relates to an optical depilation apparatus that performs depilation by irradiating skin with light from a light irradiation unit.

### BACKGROUND ART

An optical depilation apparatus in which hair papilla is heated by transferring light energy emitted by a laser diode or a flashlamp to the hair, thereby making it difficult for nutrients to reach the hair, is known as a device for conducting depilation. In such an optical depilation apparatus, the skin with hairs is required to be irradiated with light from the direction perpendicular to the skin. Accordingly, in the optical depilation apparatus disclosed in Patent Document 1 or Patent Document 2 below, a plurality of contact sensors are disposed in positions surrounding the laser beam irradiation portion of the distal end of the apparatus, the optical depilation apparatus is determined to have a perpendicular posture when the contact sensors detect contact with the skin, and the laser beam irradiation is then started.

However, in the above-described optical depilation apparatus, safety measures taken to prevent the light from leaking to the outside are insufficient. In a case where a flashlamp generating flashes is used as the light source, the flashes easily leak to the outside and can enter the eyes of the user or other people. Furthermore, the flashes can be unintentionally and erroneously emitted when the optical depilation apparatus is maintained and the flashes can enter the eyes of the user or other people. Therefore, more reliable safety measures are needed. Patent Document 1: Japanese Unexamined Patent Publication No. 2005-278724.

Patent Document 2: Japanese Unexamined Patent Publication No. 2006-525036.

### DISCLOSURE OF THE INVENTION

It is an object of the present invention to provide an optical depilation apparatus that resolves the above-described problems.

It is another object of the present invention to provide an optical depilation apparatus of high safety, in which the light irradiating the skin during the depilation treatment can be prevented from leaking and entering the eyes, maintenance can be facilitated, and the light for depilation can be prevented from being emitted at an unintended time and entering the eyes.

In order to attain the above-described objects, the optical depilation apparatus according to the present invention is an optical depilation apparatus for performing depilation by irradiating skin with light, comprising: an apparatus body; a light source unit that is provided at the apparatus body and capable of emitting light for irradiating skin; a light shielding pipe that has a skin contact surface at an end surface on a side opposite to the apparatus body in an axial direction of the light shielding pipe and is attached detachably to the apparatus body so as to surround a portion of the apparatus body which radiates light emitted from the light source unit; and a control circuit that disables the emission of light from the light source unit in a state in which the light shielding pipe is not attached to the apparatus body.

### BRIEF DESCRIPTION OF THE DRAWINGS

FIG. 1 is a cross-sectional view illustrating schematically a state in which a light shielding pipe of the optical depilation apparatus of one embodiment of the invention is in the uppermost position.

FIG. 2 is a cross-sectional view illustrating schematically a state in which the light shielding pipe of the optical depilation apparatus of one embodiment of the invention is in the downmost position.

FIG. 3 is a front view of the optical depilation apparatus shown in FIG. 1.

FIG. 4 is a side view of the optical depilation apparatus shown in FIG. 1.

FIG. 5 is a plan view of the optical depilation apparatus shown in FIG. 1.

FIG. 6 is a front view of the optical depilation apparatus illustrating a state in which the light shielding pipe in the optical depilation apparatus shown in FIG. 1 is depressed down and an irradiation switch is exposed.

FIG. 7 is a side view of the optical depilation apparatus illustrating a state in which the light shielding pipe in the optical depilation apparatus shown in FIG. 1 is depressed down and the irradiation switch is exposed.

FIG. 8 is a cross-sectional view illustrating the structure of the irradiation switch.

FIG. 9 is a side view illustrating the structure of the irradiation switch.

FIG. 10 is a circuit block diagram illustrating schematically an example of control circuit.

FIG. 11 is a circuit block diagram illustrating schematically another example of control circuit.

FIG. 12 is a circuit block diagram illustrating schematically yet another example of control circuit.

### BEST MODE FOR CARRYING OUT THE INVENTION

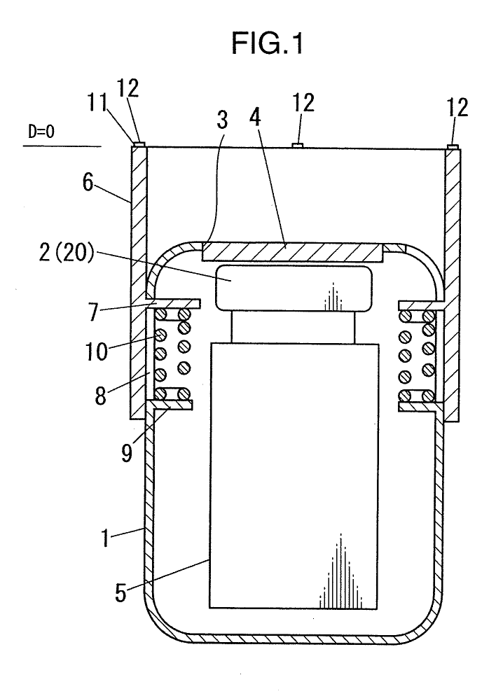

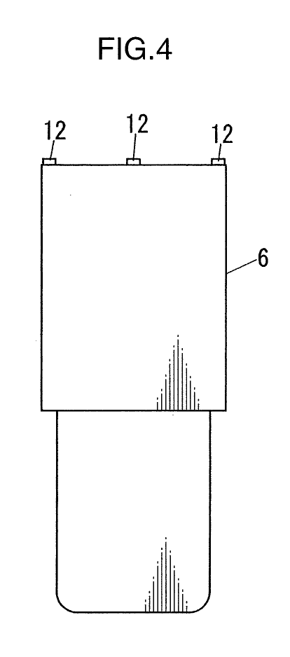

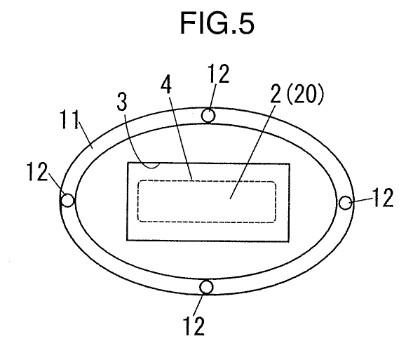

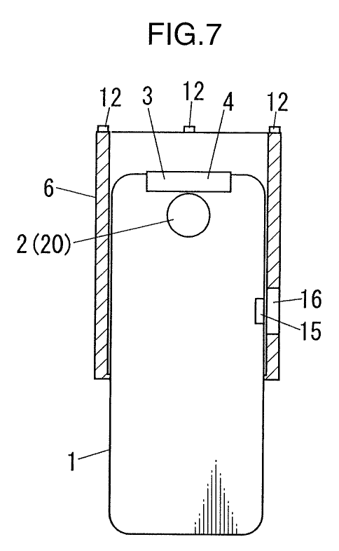

The embodiments of the present invention will be described below with reference to the appended drawings. FIGS. 1 to 7 illustrate schematically the entire structure of the optical depilation apparatus according to one embodiment of the invention.

The optical depilation apparatus of the present embodiment is provided with a box-shaped apparatus body 1 that can be grasped by one hand. A flashlight 2 that is used as a light source unit 20 is accommodated in the apparatus body 1. The flashlight 2 is composed of a xenon lamp. A light irradiation port 3 that links the inside of the apparatus body 1 with the outside is opened in a position close to the flashlight 2 at the distal end of the apparatus body 1. This light irradiation port 3 is disposed so as to be opposite the flashlight 2. A transparent lamp cover 4 is fitted in the light irradiation port 3. The lamp cover 4 is provided at the apparatus body 1 so that the outer surface of the lamp cover 4 is exposed. The flashlight 2 emits pulse-shaped flashes and these flashes are transmitted through the lamp cover 4 and irradiated to the outside of the apparatus body 1. Thus, the light emitted by the flashlight 2 is emitted from the apparatus body 1 at the lamp cover 4.

The optical depilation apparatus is also provided with a light shielding pipe 6 detachably attached in a location around the light irradiation port 3 on the outer circumference of the apparatus body 1. The light shielding pipe 6 is formed as an elliptical pipe by using a flexible elastic material such as rubber or elastomer. Further, the light shielding pipe 6 is floatably attached to the apparatus 1. The “floatable state” as referred to herein means a state in which the light shielding pipe 6 is free to move back and forth in the axial direction, while the light shielding pipe 6 is being biased in the axial direction to the side opposite to the apparatus body 1. The direction on the side opposite to the apparatus body 1 in the axial direction of the light shielding pipe 6, that is, the direction forward along the light irradiation direction will be referred to hereinbelow as “upward”, and the direction on the side the apparatus body 1 in the axial direction of the light shielding pipe 6, that is, the direction rearward along the light irradiation direction will be referred to hereinbelow as “downward”.

In the example shown in the figure, a mechanism composed of a linking protruding portion 7, a linking port 8, a receiving portion 9, and a spring member 10 is provided as an attachment mechanism for floatably attaching the light shielding pipe 6 to the apparatus body 1. The linking protruding portion 7 extends inward from the inner circumferential surface of the light shielding pipe 6. The linking port 8 is opened at the side wall of the apparatus body 1 and serves to receive movably in the vertical direction the linking protruding portion 7 and couple the light shielding pipe 6 to the apparatus body 1 so that the light shielding pipe can move back and forth in the vertical direction. The receiving portion 9 is provided at the inner circumferential surface of the apparatus body 1 in a position adjacent to the lower end edge of the linking port 8. The spring member 10 is composed of a compression coil spring inserted between the receiving portion 9 and linking protruding portion 7. The attachment of the light shielding pipe 6 to and detachment of the light shielding pipe 6 from the apparatus body 1 are conducted, for example, by pulling out the apparatus body 1 from the light shielding pipe 6 and inserting the apparatus body 1 into the light shielding pipe 6, while causing certain elastic deformation of the linking protruding portion 7 of the light shielding pipe 6.

The light shielding pipe 6 has a skin contact surface 11 as shown in FIGS. 1 to 5. The skin contact surface 11 is pushed into direct contact with a skin S of a human body and composed of an end surface on the side opposite to the apparatus body 1 in the axial direction of the light shielding pipe 6, that is, of an upper end surface of the light shielding pipe 6. A plurality of contact sensors 12 are provided with an equal spacing in the circumferential direction on the skin contact surface 11. A control circuit 5 determines that the skin S of the human body is in intimate contact with the distal end portion of the light shielding pipe 6 and enables the emission of light from the flashlight 2 only in a case where all the contact sensors 12 detect contact with the skin S.

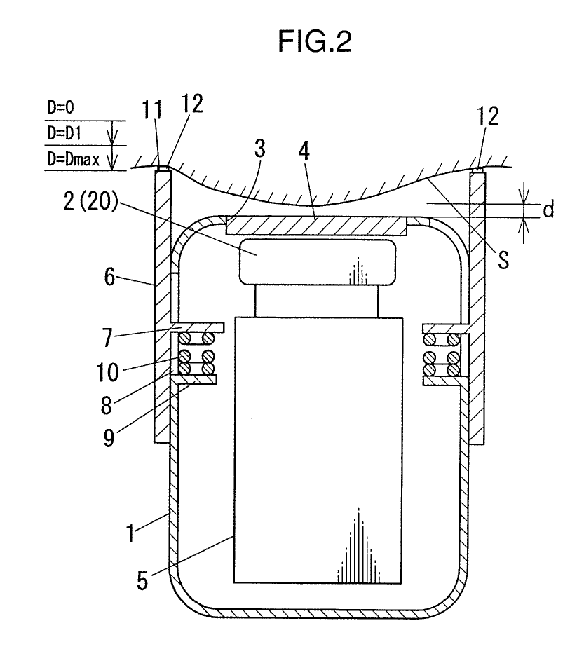

FIG. 1 illustrates a state in which the light shielding pipe 6 is in the uppermost position in the optical depilation apparatus, that is, a state in which a depression amount of the light shielding pipe 6 with respect to the apparatus body 1 is zero (D=0). FIG. 2 illustrates a state in which the light shielding pipe 6 is in the lowermost position in the optical depilation apparatus, that is, a state in which the depression amount D is equal to the maximum depression amount Dmax. The vertical size of the light shielding pipe 6 is set such that the skin contact surface 11 is positioned above the light irradiation port 3 of the apparatus body 1 in both states. Further, the vertical size of the light shielding pipe 6 is set such that a gap d is ensured between the light irradiation port 3 and the skin S that bulges downward by the pressing force of the skin contact surface 11 even in a state in which the light shielding pipe 6 has been depressed to the maximum depression amount Dmax as shown in FIG. 2.

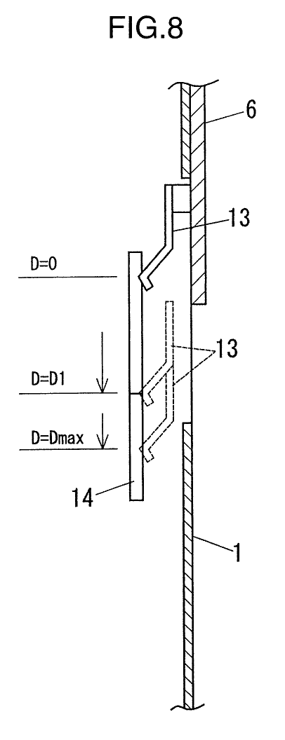

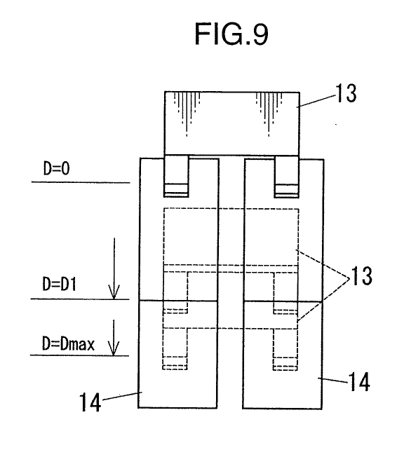

As shown in FIGS. 8 and 9, a switch spring 13 is fixed to the inner circumferential surface of the light shielding pipe 6, and a pair of connection terminals 14 are incorporated in the apparatus body 1. The switch spring 13 comes into contact with the pair of connection terminals 14 of the apparatus body 1 and enables electric conduction with the connection terminals 14 only in a case where the downward depression amount D of the light shielding pipe 6 with respect to the apparatus body 1 is equal to or greater than a set depression amount D1 that has been set in advance (0<D1<Dmax). Where it is possible to conduct electricity between the switch spring 13 and connection terminals 14, the control circuit 5 enables light emission from the flashlight 2. Light emission from the flashlight 2 is enabled by the control circuit 5 on condition that the switch spring 13 and the connection terminals 14 are in contact. Therefore, the flashlight 2 can emit light only in a case where the light shielding pipe 6 is attached to the apparatus body 1 in a predetermined position, and the flashlight 2 is unable to emit light in a state where the light shielding pipe 6 is detached from the apparatus body 1.

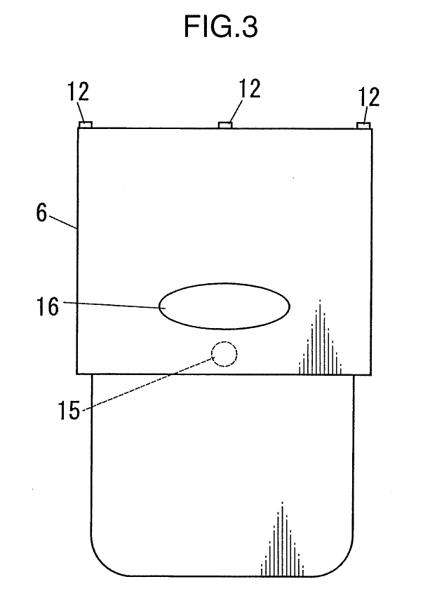

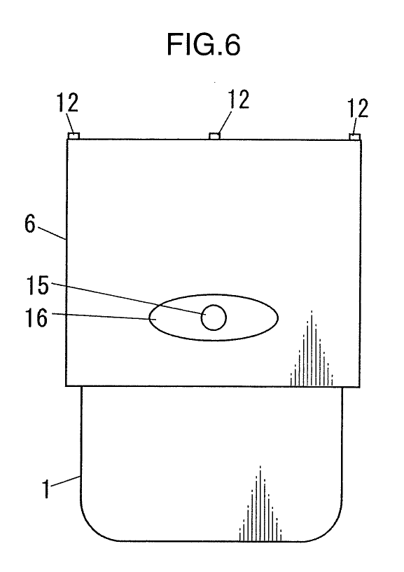

As shown in FIGS. 3 and 6, an irradiation switch 15 is provided in the center of the front side of the outer circumferential surface of the apparatus body 1. The irradiation switch 15 enables the user to ON/OF operate manually the emission of light from the flashlight 2. An operation port 16 is formed at the light shielding pipe 6 so as to expose the irradiation switch 15 when the light shielding pipe 6 is depressed downward to a certain amount. The operation port 16 is provided in a position such that the irradiation switch 15 can be exposed when the switch spring 13 and the connection terminals 14 come into contact. Further, the operation port 16 is formed to have a size such that makes it possible to expose the irradiation switch 15 at least when the depression amount D of the light shielding pipe 6 is within a range of D1 to Dmax. The control circuit 5 enables light emission from the flashlight 2 only in a case where the user continuously pushes the irradiation switch 15 exposed from the operation port 16 or where the irradiation switch is fixed in a pushed-down state.

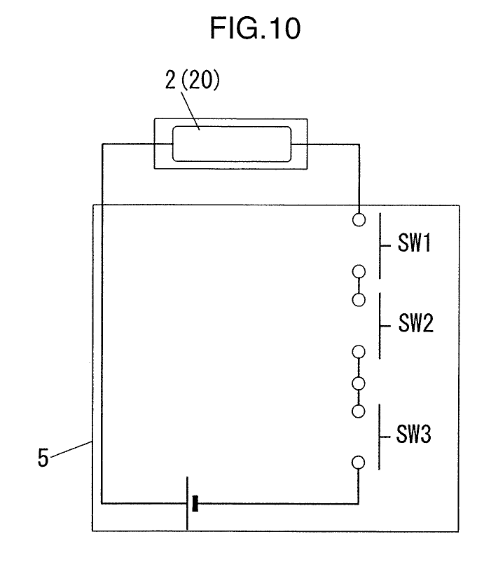

As shown schematically in FIG. 10, the control circuit 5 of the present embodiment is provided with a triple switch circuit. The triple switch circuit is configured by connecting a first switch SW1, a second switch SW2, and a third switch SW3 in series.

The first switch SW1 is configured with the switch spring 13 and the connection terminals 14 (see FIGS. 8 and 9). The first switch SW1 is switched ON when the depression amount D of the light shielding pipe 6 with respect to the apparatus body 1 is equal to or greater than the set depression amount D1 in a state in which the light shielding pipe 6 is attached to the apparatus body 1, but switched OFF when the light shielding pipe 6 is not attached to the apparatus body 1 and when the depression amount of the light shielding pipe 6 with respect to the apparatus body 1 is less than the set depression amount D1 even when the light shielding pipe 6 is attached to the apparatus body 1.

The second switch SW2 is electrically connected to each contact sensor 12 so as to be switched ON when all the contact sensors 12 provided at the distal end of the light shielding pipe 6 detect contact with the skin S. The second switch SW2 is OFF in a case where at least one of a plurality of contact sensors 12 does not detect contact with the skin S.

The third switch SW3 is ON when the user pushes the irradiation switch 15 disposed at the apparatus body 1 and OFF when the irradiation switch 15 is not pushed.

The control circuit 5 supplies power to the flashlight 2 and causes the flashlight 2 to emit pulsed light only in a case where the following conditions are satisfied: all the switches SW1, SW2, SW3 are ON, that is, the depression amount D of the light shielding pipe 6 with respect to the apparatus body 1 is equal to or greater than the set depression amount D1 in a state in which the light shielding pipe 6 is attached to the apparatus body 1, the distal end of the light shielding pipe 6 is in intimate contact with the skin S and all the contact sensors 12 are ON, and the user pushes down the irradiation switch 15 of the apparatus body 1. In other words, in a case where the light shielding pipe 6 is not attached to the apparatus body 1 and where the depression amount of the light shielding pipe 6 with respect to the apparatus body 1 is less than the set depression amount D1 even when the light shielding pipe 6 is attached to the apparatus body 1, the first switch SW1 is OFF and, therefore, the control circuit 5 interrupts power supply to the flashlight 2, thereby disabling light emission from the flashlight 2. Further, in a case where at least one contact sensor 12 provided at the skin contact surface 11 of the light shielding pipe 6 does not detect contact with the skin S, the second switch SW2 is OFF and, therefore, the control circuit 5 interrupts power supply to the flashlight 2, thereby disabling light emission from the flashlight 2. In a case where the irradiation switch 15 is not pushed, the third switch SW3 is OFF and, therefore, the control circuit 5 interrupts power supply to the flashlight 2, thereby disabling light emission from the flashlight 2.

When the optical depilation apparatus of the present invention having the above-described configuration is used by the user, the user grasps the apparatus body 1 with one hand, brings the skin contact surface 11 of the light shielding pipe 6 into contact with the skin S, and positions the skin S in the treatment object zone inside the region surrounded by the ring-shaped skin contact surface 11. In this state, the user further pushes the apparatus body 1 toward the skin S. As a result, the light shielding pipe 6 is pushed down against the biasing force of the spring member 10, and the biasing force applied from the spring member 10 upward to the light shielding pipe 6 increases accordingly. Due to the biasing force of the spring member 10, the skin contact surface 11 of the light shielding pipe 6 is brought into intimate contact with the skin S over the entire periphery. In this state, even if the user somewhat moves the apparatus body 1, the light shielding pipe 6 that is in the floating state maintains the state of intimate contact with the skin S, while following the movement of the apparatus body 1 and moving up and down.

When the user then manually switches ON the irradiation switch 15 exposed from the operation port 16 of the light shielding pipe 6 in a state in which the depression amount D of the light shielding pipe 6 is equal to or greater than the set depression amount D1 and every contact sensor 12 detects contact with the skin S, the flashlight 2 emits pulsed light, the light passes through the light irradiation port 3, that is, through the lamp cover 4, and the skin S in the treatment object zone is irradiated thereby. The depilation treatment of the skin S in the treatment object zone is conduced by such irradiation. In this case, because the skin contact surface 11 of the light shielding pipe 6 maintains the state of pressure contact with the skin S over the entire periphery of the skin contact surface 11, the flash irradiated through the light irradiation port 3 is prevented from leaking to the outside, and the flash cannot enter the eyes of the user or other people. Further, because the light shielding pipe 6 is formed from a flexible elastic material such as rubber, the state of intimate contact of the skin contact surface 11 of the light shielding pipe 6 against the skin S can be ensured more effectively.

Further, because the switch for ON/OFF switching the flashlight 2 is the triple switch of the above-described configuration, unless the user intentionally operates the irradiation switch 15 in a state in which the skin contact surface 11 of the light shielding pipe 6 is brought into intimate contact with the skin S by a sufficient pressure force over the entire periphery, the emission of light from the flashlight 2 is disabled and safety is further increased.

Further, because the light shielding pipe 6 is detachably attached to the apparatus body 1, when maintenance is conducted, for example, when the lamp cover 4 is removed and the flashlight 2 is replaced, the light shielding pipe 6 can be detached from the apparatus body 1 to facilitate the maintenance. In addition, in the present embodiment, the emission of light from flashlight 2 is enabled only in a case where the switch spring 13 of the light shielding pipe 6 and the connection terminals 14 of the apparatus body 1 are in contact, that is, when the light shielding pipe 6 is attached to the apparatus body 1. Therefore, as described above, light irradiation can be prevented in a state in which the light shielding pipe 6 is detached from the apparatus body 1, and flashes of the flashlight 2 can be prevented from entering the eyes.

All the features of the embodiment disclosed above are exemplary and should not be construed as limiting. The scope of the present invention is represented by the claims, rather than limited to the above-described embodiment, and includes all the variations that are equivalent to the claims in meaning and scope.

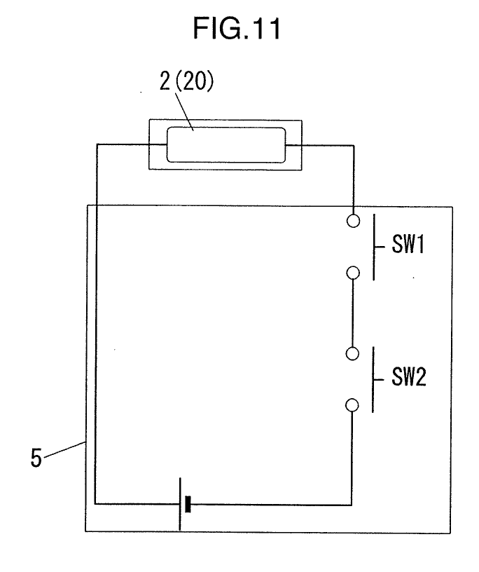

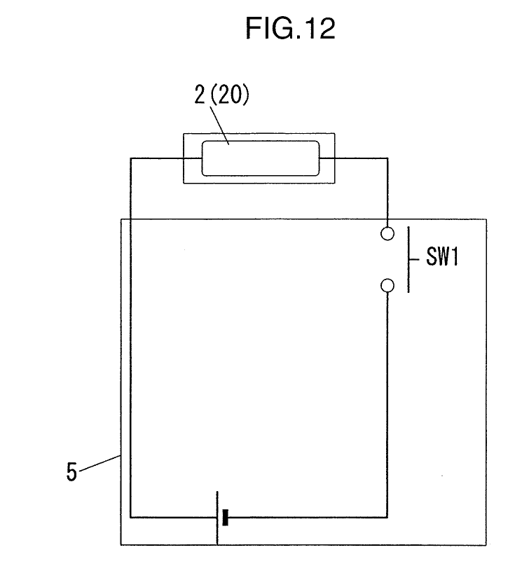

For example, the switch circuit in the control circuit 5 may be a double switch circuit in which the first switch SW1 and second switch SW2 are connected in series, as shown in FIG. 11, or a switch circuit composed only of the first switch SW1, as shown in FIG. 12, rather than the triple switch circuit. In the case where the double switch circuit shown in FIG. 11 is provided in the control circuit 5, power is supplied to the flashlight 2 and the flashlight 2 emits pulsed light at a stage at which the depression amount D of the light shielding pipe 6 with respect to the apparatus body 1 becomes equal to or greater than the set depression amount D1 in a state in which the light shielding pipe 6 is attached to the apparatus body 1, and all the contact sensors 12 are ON due to the intimate contact of the distal end of the light shielding pipe 6 with the skin S. Further, in a case where the switch circuit shown in FIG. 12 is provided in the control circuit 5, power is supplied to the flashlight 2 and the flashlight 2 emits pulsed light at a stage at which the light shielding pipe 6 is attached to the apparatus body 1 and the depression amount D of the light shielding pipe 6 with respect to the apparatus body 1 becomes equal to or greater than the set depression amount D1.

A switch circuit located in the control circuit 5 may also be a double switch circuit in which the first switch SW1 and third switch SW3 are connected in series. Thus, the second switch SW2 may be omitted from the above-described triple switch circuit located in the control circuit 5. In this case, power is supplied to the flashlight 2 and the flashlight 2 emits pulsed light at a stage at which the depression amount D of the light shielding pipe 6 with respect to the apparatus body 1 becomes equal to or greater than the set depression amount D1 in a state in which the light shielding pipe 6 is attached to the apparatus body 1 and the irradiation switch 15 of the apparatus body 1 is pushed.

Further, the light source unit 20 is not limited to the flashlight 2 composed of the xenon light and may be the flashlight 2 having a different structure. Alternatively, the light source unit 20 may be something that emits laser light by using a laser diode or the like.

### Essence of the Embodiment

The above-described embodiment is summarized below.

Thus, the optical depilation apparatus of the present embodiment is an optical depilation apparatus for performing depilation by irradiating skin with light, including: an apparatus body; a light source unit that is provided at the apparatus body and capable of emitting light for irradiating the skin; a light shielding pipe that has a skin contact surface at an end surface on the side opposite to the apparatus body in an axial direction of the light shielding pipe and is attached detachably to the apparatus body so as to surround a portion of the apparatus body which radiates light emitted from the light source unit; and a control circuit that disables the emission of light from the light source unit in a state in which the light shielding pipe is not attached to the apparatus body.

With such a configuration, the skin surrounded by the light shielding pipe can be irradiated with light for depilation that is emitted from the light source unit in a state in which the skin contact surface of the light shielding pipe is in contact with the skin. In this case, the light irradiated from the apparatus body can be prevented from leaking to the outside by the light shielding body surrounding the portion of the apparatus body which radiates light emitted from the light source unit, and the light can be prevented from entering the eyes of the user or other people. Furthermore, with this configuration, because the light shielding pipe is detachably attached to the apparatus body, when maintenance of the apparatus body is performed, the light shielding pipe can be detached from the apparatus body, thereby facilitating the maintenance of the apparatus body. In addition, in this configuration, because the control circuit disables light emission from the light source unit in a state in which the light shielding pipe is not attached to the apparatus body, the unintended light emission can be prevented and the light can be prevented from entering the eyes of the user or other people during maintenance in which the light shielding pipe is detached from the apparatus body. Therefore, such a configuration makes it possible to obtain a highly safe optical depilation apparatus in which the light used to irradiate the skin during depilation can be prevented from leaking and entering the eyes, maintenance can be facilitated, and the light for depilation can be prevented from being emitted unintentionally and entering the eyes.

In the optical depilation apparatus, it is preferred that the control circuit has a first switch that is switched OFF in a state in which the light shielding pipe is not attached to the apparatus body and interrupt power supply to the light source unit when the first switch is OFF.

With such a configuration, it is possible to constitute a specific structure of the control circuit that disables the emission of light from the light source unit when the light shielding pipe is not attached to the apparatus body.

In this case, it is preferred that the first switch is ON when the light shielding pipe is attached to the apparatus body and a depression amount of the light shielding pipe with respect to the apparatus body is equal to or greater than a set depression amount that has been set in advance.

With such a configuration, the light source unit can emit light when the user presses the skin contact surface of the light shielding pipe attached to the apparatus body against the skin and the depression amount of the light shielding pipe with respect to the apparatus body becomes equal to or greater than a set depression amount. Therefore, the unintentional emission of light when the optical depilation apparatus is not used can be prevented more reliably. Therefore, a more safe optical depilation apparatus can be obtained.

Further in this case, it is preferred that the optical depilation apparatus has a plurality of contact sensors that are provided with a predetermined spacing in a circumferential direction at the skin contact surface; and the control circuit has a second switch that is turned ON when all the contact sensors detect contact with the skin, interrupts power supply to the light source unit when the second switch is turned OFF, and supplies power to the light source unit when the first switch is turned ON and the second switch is turned ON.

With such a configuration, the light source unit cannot emit light unless all the contact sensors provided with a predetermined spacing in a circumferential direction at the skin contact surface detect contact with the skin. Therefore, it is possible to prevent the light from leaking to the outside through a gap that is formed between the skin contact surface and the skin, when the optical depilation apparatus is used. Furthermore, with such a configuration, the light source unit can emit light only when the depression amount of the light shielding pipe with respect to the apparatus body is equal to or greater than a set depression amount and additionally when all the contact sensors provided at the skin contact surface of the light shielding pipe detect contact with the skin. Therefore, unintentional irradiation with light can be prevented even more reliably. As a result, a more safe optical depilation apparatus can be obtained.

In addition, in this case, it is preferred that the optical depilation apparatus has an irradiation switch that is provided at the apparatus body and that the control circuit has a third switch that is turned ON when a user pushes the irradiation switch, interrupts power supply to the light source unit when the third switch is turned OFF, and supplies power to the light source unit when the first switch, the second switch, and the third switch are all turned ON.

With such a configuration, the light source unit cannot emit light unless the user pushes the irradiation switch. Therefore, unintentional emission of light can be prevented even more reliably. Furthermore, with such a configuration, the light source unit can emit light only when the depression amount of the light shielding pipe with respect to the apparatus body is equal to or greater than a set depression amount, all the contact sensors provided at the skin contact surface of the light shielding pipe detect contact with the skin, and additionally the user has pushed the irradiation switch. Therefore, an even safer optical depilation apparatus can be obtained.

In the configuration in which the first switch is turned ON when the depression amount of the light shielding pipe with respect to the apparatus body is equal to or greater than the set depression amount that has been set in advance, it is preferred that the optical depilation apparatus has an irradiation switch that is provided at the apparatus body and that the control circuit has a third switch that is turned ON when a user pushes the irradiation switch, interrupts power supply to the light source unit when the third switch is turned OFF, and supplies power to the light source unit when the first switch is turned ON and the third switch is turned ON.

With such a configuration, the light source unit cannot emit light unless the user pushes the irradiation switch. Therefore, unintentional emission of light can be prevented even more reliably. Furthermore, with such a configuration, the light source unit can emit light only when the depression amount of the light shielding pipe with respect to the apparatus body is equal to or greater than a set depression amount and additionally the user has pushed the irradiation switch. Therefore, an even safer optical depilation apparatus can be obtained.

1. An optical depilation apparatus for performing depilation by irradiating skin with light, comprising:
an apparatus body;
a light source unit that is provided at the apparatus body and capable of emitting light for irradiating skin;
a light shielding pipe that has a skin contact surface at an end surface on a side opposite to the apparatus body in an axial direction of the light shielding pipe and is attached detachably to the apparatus body so as to surround a portion of the apparatus body which radiates light emitted from the light source unit; and
a control circuit that disables the emission of light from the light source unit in a state in which the light shielding pipe is not attached to the apparatus body.

2. The optical depilation apparatus according to claim 1, wherein the control circuit has a first switch that is switched OFF in a state in which the light shielding pipe is not attached to the apparatus body and interrupts power supply to the light source unit when the first switch is OFF.

3. The optical depilation apparatus according to claim 2, wherein the first switch is ON when the light shielding pipe is attached to the apparatus body and a depression amount of the light shielding pipe with respect to the apparatus body is equal to or greater than a set depression amount that has been set in advance.

4. The optical depilation apparatus according to claim 3, comprising a plurality of contact sensors that are provided with a predetermined spacing in a circumferential direction at the skin contact surface, wherein
the control circuit has a second switch that is turned ON when all the contact sensors detect contact with the skin, interrupts power supply to the light source unit when the second switch is turned OFF, and supplies power to the light source unit when the first switch is turned ON and the second switch is turned ON.

5. The optical depilation apparatus according to claim 4, further comprising an irradiation switch that is provided at the apparatus body, wherein
the control circuit has a third switch that is turned ON when a user pushes the irradiation switch, interrupts power supply to the light source unit when the third switch is turned OFF, and supplies power to the light source unit when the first switch, the second switch, and the third switch are all turned ON.

6. The optical depilation apparatus according to claim 3, comprising an irradiation switch that is provided at the apparatus body, wherein
the control circuit has a third switch that is turned ON when a user pushes the irradiation switch, interrupts power supply to the light source unit when the third switch is turned OFF, and supplies power to the light source unit when the first switch is turned ON and the third switch is turned ON.
